(19) United States
(12) Patent Application Publication
(10) Pub. No.: US 2010/0179662 A1
(43) Pub. Date: Jul. 15, 2010

(54) MONOBLOCK CERAMIC PROSTHESIS DEVICES

(75) Inventors: Enrica Verne, Torino (IT); Chiara Vitale Brovarone, Torino (IT); Lorenza Robiglio, Torino (IT); Francesco Baino, Asti (IT)

Correspondence Address: SUGHRUE MION, PLLC, 2100 PENNSYLVANIA AVENUE, N.W., SUITE 800, WASHINGTON, DC, 20037, US

(73) Assignee: POLI DI TORINOTECNICO, Torino (IT)

(21) Appl. No.: 12/602,324

(22) PCT Filed: May 28, 2008
(86) PCT No.: PCT/IT2008/000350

§ 371 Date: Nov. 30, 2009

(30) Foreign Application Priority Data
May 29, 2007 (IT) TO2007A000373

(51) Int. Cl. A61F 2/38; A61F 2/32

(52) U.S. Cl. 623/20.32; 623/20.35; 623/22.21

(57) ABSTRACT

Single-piece prosthesis elements chosen from among acetabular cup for hip prosthesis, tibial component and femoral component for knee prosthesis, comprising a body (2) of ceramic material or ceramic matrix composite material, a bioactive porous coating (4) of glass material, glass-ceramic material or glass-ceramic matrix material applied to the surface of said body intended for anchoring with the bone tissue, where said bioactive porous coating (4) is anchored to the body surface (2) by means of a glass or glass-ceramic phase layer.

The present invention refers to a ceramic prosthesis element, having a single-piece structure provided with a bioactive glass-ceramic trabecular coating, as well as a process for its production.

In a first embodiment, the invention refers to an acetabular cup for hip prosthesis.

The hip is an articulation composed of the femur, long bone which constitutes the thigh skeleton, and the acetabulum (or cotyloid cavity), pelvis cavity which, receives the head of the femur. The object of an artificial articulation is to make a system which restores the physiological kinematics and permits supporting the loads, minimising wear and friction, and avoiding the rise of damaging reactions in the organism.

A hip prosthesis is composed of
a stem, fixed in the diaphyseal channel of the femur, always made of metal,
a femoral head, made of metal or ceramic, connected to the stem by means of conical coupling,
an acetabular cup, which is articulated on the femoral head, normally made of UHMWPE or ceramics or metal (called insert),
an acetabular shell, which rigidly encloses acetabular cup, made of metal (metal back).

Limiting our attention to the acetabular component, it is necessary to underline that, normally, such component is built according to a modular strategy, i.e. the acetabular cup (insert) is housed in the metal acetabular shell (metal back) and this permits being able to combine different materials together. For example, the insert can be composed both of polyethylene and ceramics. The combination of the different parts is usually predetermined by the size of the articulation.

However, it is known [1] that one of the main disadvantages deriving from the modular geometry consists of the high risk, after assembly, of relative mobility between the components, which irreparably leads to wear phenomena, and in some cases to failure of the prosthesis.

Another disadvantage of the modular cotyloid cavities lies in the difficulty of making small calibre prostheses (for example, those for children), since the need to use a metal shell makes it impossible to use of a ceramics insert: it would in fact be too thin to support the stresses in vivo. In this prosthesis type, therefore, it is necessary to forgo the ceramic/ceramic coupling (i.e. the ceramic prosthesis head which is articulated in an acetabular cup with ceramic insert) and opt for a metal/metal or metal/polyethylene coupling, for which there still exist wear and biocompatibility problems that make them little adapted for use in young patients.

Making a cotyloid cavity in a single piece is currently a subject of high interest.

One of the few examples of single-piece cotyloid cavity is that made and sold by Zimmer [2], where a shell of porous tantalum (Trabecular Metal®) is anchored via pressure die-casting with the insert in polyethylene. This device has numerous advantages with respect to the traditional modular cotyloid cavity with metal back (widely reported in the mentioned literature) but has the indisputable disadvantage that it can only be used with polymer inserts.

WO2007/021936 proposes a hip prosthesis including acetabular cup of ceramic material, having a ceramic substrate with low porosity, preferably composed of silicon nitride or an aluminium/zirconium material, having a porous ceramic surface coating (scaffold).

In another embodiment, the invention refers to a femur component or a tibial component of a knee prosthesis.

The knee prosthesis is conceptually a coating prosthesis, i.e. it coats the damaged surfaces, which are previously prepared to give them the shape that the prosthesis has at its interior, so as to obtain a stable fixing.

The prosthesis is therefore composed at least of two main elements: the femoral component and the tibial component. The tibial component is completed with a polymer insert, on which the femoral component is articulated. The kneecap can be preserved or prosthetic.

The problems connected with the choice of the materials most adapted for making a knee prosthesis are of two types: the dimensions, which cannot exceed those of the bone to which the prosthesis will have to be fixed, and the materials, which must be biocompatible.

The material must possess optimal mechanical prostheses for supporting the loads, but must also be easily workable. The metal materials offer an optimal response to needs tied to making a knee prosthesis. In particular, three families of metals are used:

1. stainless steels, adapted for building the tibial components that are fixed to the bone with an acrylic resin (polymethylmethacrylate—PMMA), commonly called “bone cement”;
2. cobalt-chromium based alloys, above all used for the sliding parts, like the femoral component;
3. titanium and its alloys, which are capable of binding to the bone without the need for cement and have optimal strength qualities, but are not adapted to building the sliding surfaces.

Another important material in the manufacture of a prosthesis is polyethylene. It is used in knee prostheses for coating the metal tibial plate and the prosthetic kneecap on the parts which are intended to slide on the femoral component, which in this case is always metallic (usually cobalt-chromium alloy). In some cases, the tibial plate and the kneecap can also be entirely made of polyethylene, which is then “cemented” to the bone.

Until recently, the knee prosthesis was never made with ceramic materials, because the mechanical characteristics of the latter were not sufficient for permitting the manufacture of thin, strong components. Today, thanks to the evolution of ceramics technologies, knee prostheses are beginning to be proposed with femoral component made of a ceramics compound (Biolox) coupled with a tibial component made of polyethylene, or a prosthesis characterised by a ceramic-ceramic coupling, i.e. composed of both ceramic components.

The introduction of ceramic materials, which have optimal durability characteristics with regard to the articulated surfaces, is however still limited by the poor possibility of working the inner surfaces of the components in direct contact with the bone, in order to favour their osteo-integration. It is in fact very difficult to make the surface of interest rough to the point that they can be integrated via press-fit, or coated with Plasma Spray techniques (more adapted for the metal substrates), or to ensure a good clinging of the PMMA-based cements.

One of the few examples of knee prostheses that have anchorage systems with trabecular macroporous structure (which therefore do not require coatings adapted to promote osteo-integration) is that produced and sold by Zimmer where a coating of porous tantalum (Trabecular Metal®) is anchored by pressure die-casting with the tibial plate in polyethylene. This device has numerous advantages with respect to the traditional anchoring, but has the indisputable disadvantage that it can only be used with polymer tibial plates, and thus not with the femoral components, whether metal or ceramic.

The object of the present invention is that of providing a prosthesis element with single-piece structure, chosen from among an acetabular cup for hip prosthesis and a tibial or femoral component for knee prosthesis, having improved mechanical properties, provided with bioactive characteristics that considerably increase its osteo-integration properties and which is therefore capable of stimulating bone regeneration in the implant site, also thanks to the release of ions.

Such objects are attained by means of a prosthesis element having the characteristics defined in the following claims.

One object of the invention is a single-piece prosthesis element, made of ceramic material or ceramic matrix composite material, a glass, glass-ceramic or glass-ceramic matrix composite material coating being present on its surface intended for anchoring with the bone tissue, such coating provided with a controlled and interconnected macroporosity greater than 60% by volume and with bioactive characteristics.

In this manner, optimal osteo-integration capacities are ensured of the surface in contact with the bone tissue; moreover, in the case of acetabular cup or tibial component, the single-piece structure involves no risk of malpositioning the elements, which do not require assembly before implant, and a complete mobility prevention of the elements themselves.

According to another characteristic of the invention, the aforesaid bioactive macroporous coating is anchored to the surface of the body by means of an intermediate glass or glass-ceramic phase layer.

Further characteristics and advantages of the invention will be evident from the following detailed description, carried out with reference to the attached drawings, provided as a non-limiting example, wherein:

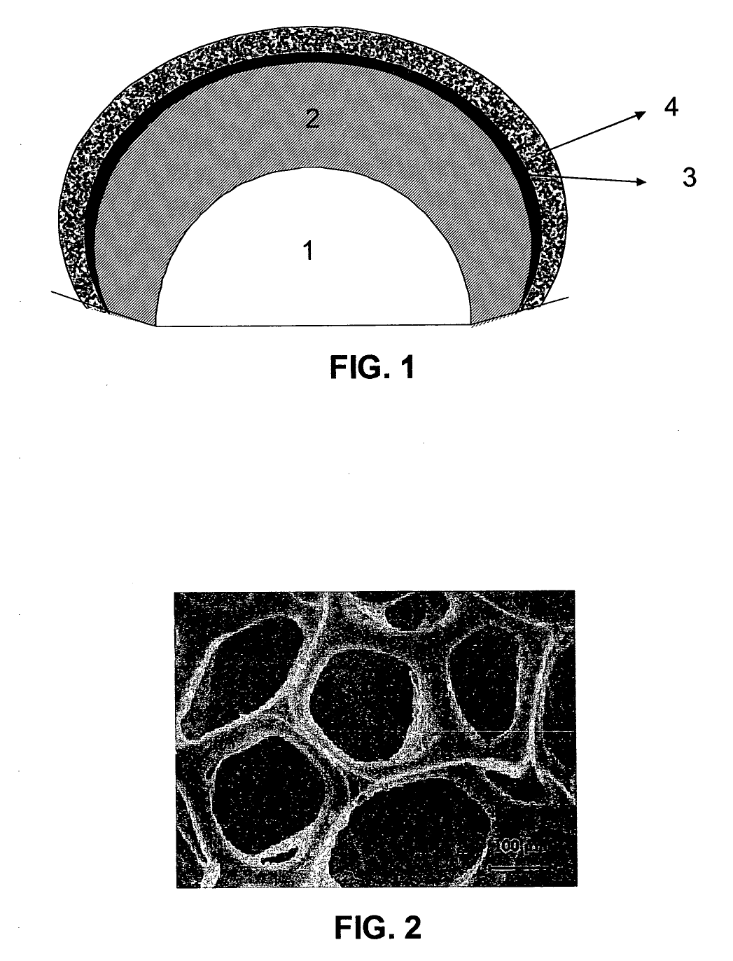

FIG. 1 is a schematic section view of an acetabular cup, object of the present invention;

FIG. 2 is a SEM micrograph of a polymer sponge used in the preparation of the macroporous coating (scaffold);

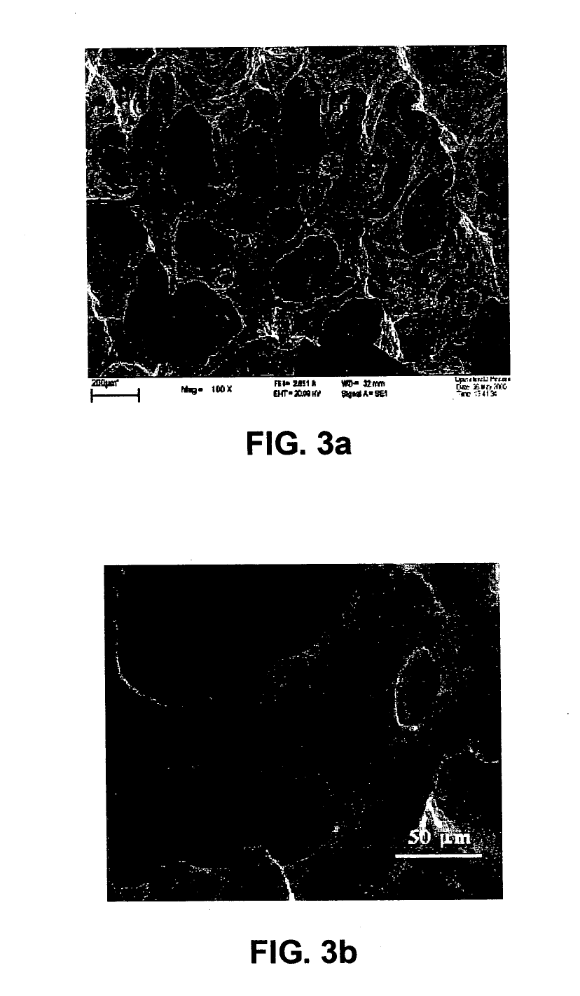

FIG. 3a is a SEM micrograph of a glass-ceramic scaffold used according to the invention;

FIG. 3b is a SEM micrograph which illustrates a detail of a trabecular structure of the scaffold;

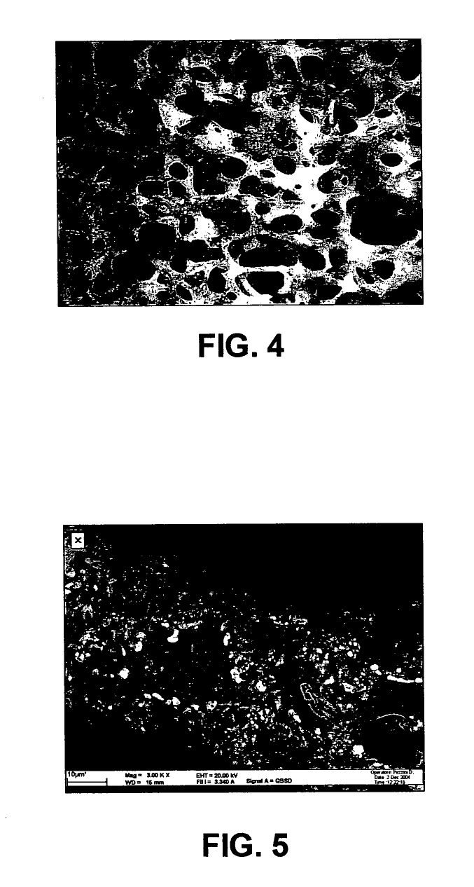

FIG. 4 is a SEM micrograph of a spongy bone portion;

FIG. 5 is a SEM micrograph of a glass-ceramic matrix scaffold, strengthened by zirconium particles;

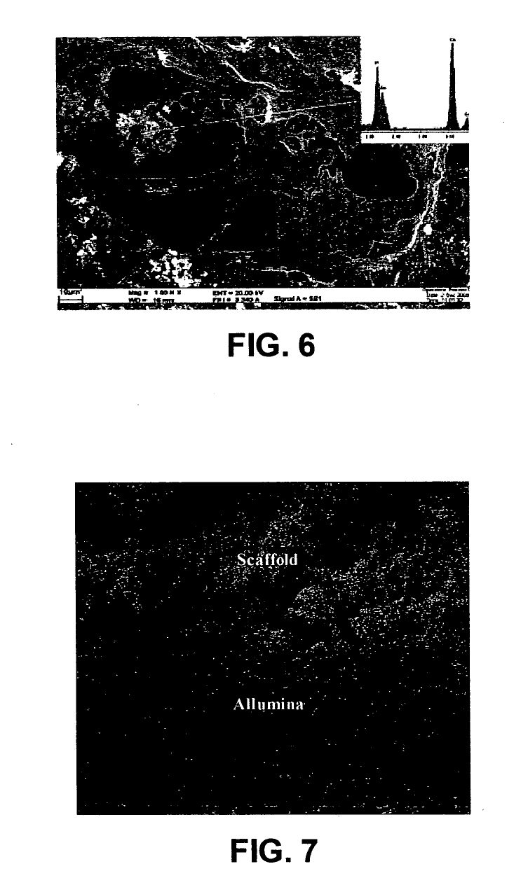

FIG. 6 is a SEM micrograph of a bioactive glass-ceramics scaffold after immersion in SBF and proliferation of osteoblasts;

FIG. 7 is a detail of a dense aluminium test piece coated with a bioactive glass-ceramic scaffold, according to the invention; and

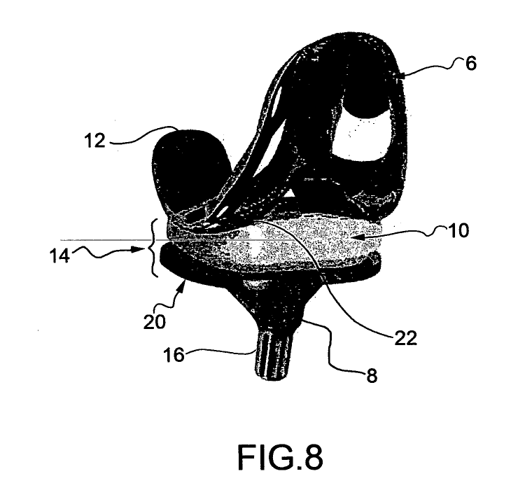

FIG. 8 is a representation of a knee prosthesis.

With reference to the schematic representation FIG. 1, an acetabular cup according to the invention comprises a body 2, defining a semi-spherical niche 1, intended to house the head of the prosthesis; a glass phase anchoring layer is indicated with 3, interposed between the body 2 and a macroporous coating layer 4 composed of bioactive glass, glass-ceramic or composite material.

The body 2 is a compact body, preferably composed of aluminium, zirconium or zirconium/aluminium composite material.

FIG. 8 illustrates a knee prosthesis with conventional structure, including a femoral component 6, a tibial component 8 and an insert 10, typically of polymer material.

According to the invention, the femoral component is a single-piece body of ceramic material or ceramic matrix composite material, and has the aforesaid macroporous coating on its surface 12, intended for anchoring with the bone tissue of the femur. The macroporous coating is bonded to the femoral component by means of a generally compact glass phase or glass-ceramic phase anchoring layer.

According to the invention, the tibial component 14 is a single-piece structure, which integrates the conventional insert 10 and the conventional tibial component 8 mentioned above in a single piece. The tibial component 14 according to the invention therefore has a tapered anchoring portion 16, intended to be inserted in the medullary cavity of the resected tibial bone and a plate portion 18 (tibial plate) with a lower surface 20, intended for the anchoring with the tibial bone tissue and an upper surface 22 that has concave seats defining articulation surfaces for the natural or prosthetic femoral component. The macroporous component according to the invention is bonded, by means of the abovementioned anchoring layer, to the lower surface 20.

The macroporous coating (scaffold) of the prosthesis elements according to the invention is preferably made through a “replica” process. The precursor is a polymer sponge, whose structure is illustrated in FIG. 2. The polymer sponge to be impregnated is produced with the shape of the coating and is over-sized, considering the shrinkage phenomena that affect the glass with which the scaffold is made during the employed heat treatment.

The polymer sponge thus this shaped is impregnated with an aqueous solution of glass or glass-ceramic powders and possibly particles of a second ceramic reinforcing phase, preferably having a granulometry of less than 10 μm; the aqueous suspension is preferably added with dispersing agents, for example polyvinyl alcohol, and left to dry at room temperature.

Through a thermal treatment at a temperature in the range of 500° C.-1200° C., the polymer sponge and the dispersing agent burn; the glass powders or glass-ceramic powders soften and sinter, generating a glass-ceramic or composite replica of the sponge. For such purpose, the glass nature of the material with which the scaffold is made, due to its softening characteristics, effectively allows incorporating a second ceramic phase so as to increase the scaffold's final mechanical properties.

The osteo-integratability of the prosthesis depends on the bioactive characteristics of the coating layer and on its macroporous morphology with trabecular structure. Bioactivity is the capacity of a material to stimulate the growth of healthy tissue in direct contact with the implant surface. This characteristic is typical of several glass and/or glass-ceramic materials, first designed by L. L. Hench in the 1970s [3] and subsequently widely studied by numerous research groups in the world. These materials, in contact with the biological fluids, undergo surface modifications adapted to promote the growth on their surface of a hydroxyapatite layer entirely similar to the mineral part of the bone. This characteristic translates into the formation of an actual chemical bond with the bone, which is then firmly anchored to the implant surface. The characteristic of glass and glass-ceramic materials, comprising those bioactive, of softening at relatively low temperatures also permits high versatility in their working and allows making coatings of thickness varying from a few dozen microns up to several millimetres, dense bone fillers, granulates, or actual macroporous scaffolds characterised by a high porosity percentage, whose size and level of interconnection are perfectly compatible with those of the human bone. The lack of risk of malpositioning and subsequent implant moving are ensured by the single-piece geometry of the prosthesis and by the osteo-integration capacities of the outer layer, which ensures a high primary and secondary stability.

The morphology of the macroporous material obtained with the replica technique is visible in FIG. 3a. A three-dimensional structure is observed characterised by, an interconnected macroporosity (preferably 65%-75% by volume) with macropores of over 100 μm size and micropores smaller than 10 μm, the latter conditions adapted for allowing a suitable supply of nutrients during the first phases of the implant and cellular colonisation, and which subsequently allow suitable vascularisation. An enlargement of the glass-ceramic trabecula can be seen in FIG. 3b, where it is also possible to observe the surface roughness that characterises the device, and which favours cellular anchoring. The trabecular morphology is very similar to that of the spongy bone, reported in FIG. 4.

The materials used according to the invention are provided with in vitro bioactivity, according to the Hench criteria. In fact, the formation of microcrystalline hydroxyapatite agglomerates can be seen via immersion in simulated physiological solutions.

From the mechanical standpoint, the obtainable compression strength is in the range of 2-15 MPa and is therefore very similar to that of the spongy bone (variable between 2 and 12 MPa). Such mechanical characteristics were obtained due to the choice of a glass composition that gives rise, during the heat treatment, to crystalline phases provided with good mechanical characteristics [4], as well as through an optimisation of the employed process conditions.

In particular, glass materials containing SiO₂ (40-60% mol.), P₂O₅ (2-6% mol.), CaO (20-30% mol.), MgO (1-20% mol.), Na₂O (10-20% mol.), K₂O (0-10% mol.) and CaF₂ (0-10% mol.) were employed by the proponents, obtaining scaffolds with mechanical strengths of up to 5 MPa. In particular, by using the following composition: 45% mol. SiO₂, 3% mol. P₂O₅, 26% CaO, 7% MgO, 15% mol. Na₂O, 4% mol. K₂O, a solid load corresponding to 25% by weight of glass, 6% by weight of PVA and the rest water, values equal to 2.5 MPa were obtained. Such values were obtained both thanks to the good mechanical characteristics of the glass-ceramic material that is obtained starting from such composition via heat treatment and thanks to the optimisation of the impregnation phases: 25% solid load, three integration cycles of 30″ duration followed by a compression of the impregnated sponge equal to 35% for a duration of 2″.

Compression strength values of up to 15 MPa were reached by adding small quantities of aluminium (Al₂O₃) to the glass composition, up to a 3% molar maximum.

The present invention also provides that the scaffold can be preferably made of a composite material, with a glass-ceramic bioactive matrix reinforced with ceramic particles such as zirconium and aluminium in order to increase the mechanical characteristics of the scaffold.

For such purpose, as an example, in FIG. 5 a detail is reported of the trabecula of a glass-ceramic scaffold reinforced with zirconium particles of micrometric size.

The high level of interconnection of the porosity permits obtaining a quick impregnation by the biological fluids (high capillarity). In addition, cell adhesion and proliferation tests have successfully shown the capacity of these materials for being suitably colonised by the osteoblasts (see FIG. 6).

The macroporous coating (scaffold) can have a thickness that varies from 0.5 to 10 millimetres as a function of the size of the ceramic body of aluminium or aluminium/zirconium composite to be coated. The glass-ceramic macroporous scaffold or glass-ceramic matrix composite macroporous scaffold can be applied to the surface of ceramic materials such as aluminium, zirconium or aluminium/zirconium composites.

The present invention also provides for a binding system of the scaffold to the body of the prosthesis element (ceramic cotyloid cavity, or prosthesis component of the knee). Such binding system is obtained by means of the use of a thin intermediate glass phase layer between the scaffold and the ceramic body; such intermediate layer is indispensable for ensuring a firm anchoring of the scaffold outside the ceramic surface.

The glass employed for such intermediate layer is preferably characterised by a linear thermal expansion coefficient in the range of 7.5-9.5×10⁻⁶/° so as to be compatible with that of aluminium (8-9×10⁻⁶/°).

In such a manner, a tensional state of residual compression at the interface with the body can be induced, thus ensuring adhesions greater than 20 MPa.

The interposition of a glass layer and its softening properties at the joining temperatures ensure a firm anchoring of the scaffold to the glass layer and consequently to the underlying ceramic body. Such joining is obtained with an ad hoc heat treatment which leads the intermediate glass layer to complete softening and the scaffold to a partial softening, thereby not altering its morphological and structural characteristics. As a function of the specific production needs of the prosthesis element, the joining of the scaffold and the ceramic body can be obtained in one of the following modes:
simultaneously upon making the intermediate glass layer;
after having made the intermediate glass layer, putting it in contact with the scaffold and carrying out a second heat treatment.

The glass usable for the coating preferably has the following composition: SiO₂ (45%-65% mol.), CaO (20%-50% mol.), B₂O₃ (0%-10% mol.), Al₂O₃ (0%-10% mol.) and can also not have bioactive characteristics, which are instead ensured by the overlying scaffold. The presence of aluminium in the glass composition can preferably be provided with the goal of increasing the compatibility between the junction state and the ceramic body.

The aforesaid intermediate layer can be applied both through heat spray techniques (plasma spray) and through traditional glazing. In particular, the latter technology is decidedly less costly than the heat spray techniques and is easily transferable to the object of the invention for making the intermediate layer. In particular, the traditional glazing of ceramic substrates provides for covering the object to be glazed with glass powders of suitable size, possibly carried by a liquid dispersing means. After having adjusted the thickness of the desired powder deposit, the possible dispersing means is made to evaporate. A subsequent heat treatment causes the melting of the powders deposited on the ceramic surface, which—during the subsequent cooling—generate a glass film adhering to the surface itself.

The anchoring layer is preferably a compact layer, but can have reduced porosity, in any case lower than that of the coating both in terms of pore size and volume.

In the scope of the invention, glass coatings are made, both on aluminium substrates and on zirconium, with or without the addition of second strengthening and/or osteo-conductive phases, reaching shear strength values at the interface on the order of 20-25 MPa, i.e. of the same magnitude, if not greater, than that of the shear strength in hydroxyapatite commonly obtained via plasma spray on titanium alloys for arthroprothesis.

The feasibility of the present invention was successfully tested by the inventors for joining bioactive glass-ceramic scaffolds, obtained with the previously described methods, on substrates of dense aluminium.

In particular, in FIG. 7, the detail is reported of a cross section of the interface between an aluminium substrate and a scaffold, joined through an intermediate glass layer where defects such as cracks or unsticking are not encountered.

With regard to the production of an acetabular cup, the present invention attains the following innovative advantages and/or characteristics:
possibility of making an osteo-integratable single-piece cotyloid cavity;
possibility of making a single-piece cotyloid cavity for prosthesis with ceramic/ceramic coupling even of small calibre;
the interposition of a glass junction layer with low thermal expansion coefficient used for connecting the scaffold to the ceramic cotyloid cavity permits attaining adhesion forces greater than 20 MPa;
the possible inclusion of aluminium in the glass composition of the intermediate layer permits increasing the compatibility between the junction layer and the ceramic cup;
bioactive characteristics are attained of the macroporous shell with trabecular structure, which considerably increases its osteo-integration, together with unusual mechanical properties for such materials (greater than 2 MPa);
possibilities of obtaining a macroporous external structure with mechanical properties even greater than 5 MPa, by using glass-ceramic materials reinforced by ceramic particles, such as zirconium and aluminium;
easy workability of the scaffold starting from the polymer sponge, by making pieces of different shapes and sizes and easy to apply to ceramic substrates;
easy technological transfer on an industrial scale.

### BIBLIOGRAPHY

1) G. Willmann, “Frettingkorrosion, ein Problem bei Hüftendoprotheses”, Praktische Orthopädie, Rheumatologie-Endoprothetik, vol. 47, 1997.
2) http://www.zimmer.com/ctl?template=CP&op=global&action=template=MP&id=1481
3) L. L. Hench, in “An Introduction to Bioceramics” edited by L. L. Hench and J. Wilson, vol. 1, World Scientific Publ., 1993, p. 41.
4) C. Vitale-Brovarone, E. Verné, L. Robiglio, P. Appendino, F. Bassi, G. Marinasso, G. Muzio, R. Canuto, Acta Biomat 3, 2007, 199.208.

1. Single-piece prosthesis element chosen from among acetabular cup for hip prosthesis, tibial component and femoral component for knee prosthesis, comprising a body (2) of ceramic material or ceramic matrix composite material, a bioactive porous coating (4) of glass material, glass-ceramic material or glass-ceramic matrix material applied to the surface of said body intended for anchoring with the bone tissue, where said bioactive porous coating (4) is anchored to the body surface (2) by means of a glass or glass-ceramic phase layer.

2. Prosthesis element according to claim 1, characterised in that said porous coating (4) has a porosity greater than 60% by volume, preferably 65%-75% by volume, referred to the total volume of the coating.

3. Prosthesis element according to claim 1, where said porous coating (4) has macropores of size greater than 100 μm and micropores of size less than 10 μm.

4. Prosthesis element according to claim 1, characterised in that said porous coating (4) is a glass comprising:

| | | |
|---|---|---|
| SiO₂ | 40%-60% | in moles |
| P₂O₅ | 2%-6% | in moles |
| CaO | 20%-30% | in moles |
| MgO | 1%-20% | in moles |
| Na₂O | 10%-20% | in moles |
| K₂O | 0%-10% | in moles, preferably 0.5%-10% in moles |
| Al₂O₃ | 0%-3% | in moles, preferably 1%-3% in moles. |

5. Prosthesis element according to claim 1, characterised in that said porous coating (4) is a composite material, with glass-ceramic bioactive matrix, reinforced by ceramic particles chosen from among zirconia and alumina.

6. Prosthesis element according to claim 1, characterised in that said porous coating (4) has a thickness in the range of 0.5-10 mm.

7. Prosthesis element according to claim 1, characterised in that said body (2) is formed by an alumina, zirconia or a zirconia/alumina composite material.

8. Prosthesis element according to claim 1, characterised in that said anchoring layer (3) is a glass layer having a linear thermal expansion coefficient in the range of 7.5-9.5×10⁻⁶/°.

9. Prosthesis element according to claim 1, characterised in that said anchoring layer (3) is a glass containing:

| | |
|---|---|
| SiO₂ | 45%-65% in moles |
| CaO | 20%-50% in moles |
| B₂O₃ | 0%-10% in moles |
| Al₂O₃ | 0%-10% in moles. |

10. Prosthesis element according to claim 1, characterised in that it consists of the femoral component (6) of a knee prosthesis, wherein said porous coating is applied to its surface (12) intended for anchoring to the bone tissue of the femur.

11. Prosthesis element according to claim 1, characterised in that it consists of the tibial component (14) of a knee prosthesis, said tibial component being a single-piece structure comprising an anchoring portion (16) intended to be inserted in the medullary cavity of the resected tibial bone and a plate portion (18) having concave seats that define articulation surfaces for the natural or prosthetic femoral component, wherein said porous coating is applied, by means of said anchoring layer, to the lower surface (20) of said plate portion, intended to being placed in contact with the tibial tissue.

12. Prosthesis element according to claim 1, characterised in that it is composed of an acetabular cup.

13. Process for producing a prosthesis element according to claim 1, characterised in that said porous coating (4) of glass, glass-ceramic or glass-ceramic matrix composite material is previously obtained through a replica process starting from a polymer sponge, and is anchored to said insert (2) by means of an intermediate glass layer (3).

14. Process according to claim 13, where the replica process comprises the operations of:
prearranging a polymer sponge, shaped according to the shape of said coating layer (2);
impregnating said polymer sponge with an aqueous suspension of glass or glass-ceramic powders optionally containing a second ceramic reinforcing phase and containing dispersing agents;
subjecting to heat treatment at temperatures in the range of 500° C.-1200° C. in order to cause the combustion of said polymer sponge and related dispersing agent, so to generate a glass, glass-ceramic or composite replica of said sponge.
